(19) United States
(12) Patent Application Publication
(10) Pub. No.: US 2006/0144408 A1
(43) Pub. Date: Jul. 6, 2006

(54) Micro-catheter device and method of using same

(76) Inventor: Steven J. Ferry, Excelsior, MN (US)

Correspondence Address: HARNESS, DICKEY, & PIERCE, P.L.C, 7700 BONHOMME, STE 400, ST. LOUIS, MO, 63105, US

(21) Appl. No.: 11/186,407

(22) Filed: Jul. 21, 2005

Related U.S. Application Data
(60) Provisional application No. 60/590,743, filed on Jul. 23, 2004.

(51) Int. Cl. A61B 19/00

(52) U.S. Cl. 128/899; 600/431

(57) ABSTRACT

The micro-catheter has regions of different flexibility along its length, and has a magnetic element responsive to an applied magnetic field disposed in the distal end. The magnetic field may be applied with at least one stationary or at least one moveable magnet external to the subject body. The distal end of the micro-catheter is sufficiently flexible and the tip is sized such that the tip can bend or deflect at least 50 degrees when subjected to a magnetic field of as low as 0.1 Tesla and more preferably as low as 0.08 Tesla having a reference angle 90 degrees relative to the orientation of the distal end of the catheter.

### CROSS-REFERENCE TO RELATED APPLICATIONS

This application claims the benefit of U.S. Provisional Patent Application Ser. No. 60/590,743, filed Jul. 23, 2004. The disclosure of the above-referenced application is incorporated herein by reference.

### FIELD OF THE INVENTION

This invention relates to catheters, and more particularly to flexible micro-catheters that may be magnetically steered within the body.

### BACKGROUND OF THE INVENTION

Magnetic catheters are catheters provided with a magnetically responsive element by which the distal end of the catheter can be navigated, or oriented by the application of a magnetic field. Various magnetic surgery systems have been developed to create a magnetic field in a selected direction in an operating region in a subject's body to orient a magnetic medical device in body. On example of such system is disclosed in U.S. Pat. No. 6,241,671, issued Jun. 5, 2001, for Open Field System for Magnetic Surgery, the disclosure of which is incorporated herein by reference. More recently, magnetic surgery systems have been developed employing compound permanent magnets. These Magnetic Surgery Systems (MSS) allow for a less-invasive method of navigating medical devices in the body for diagnostic and therapeutic procedures.

Various devices have been developed for use with magnetic surgery system. Such an apparatus and a method for navigating the apparatus is disclosed in U.S. Pat. No. 6,015,414, issued Jan. 18, 2000, for Method and Apparatus for Magnetically Controlling Motion Direction of a Mechanically Pushed Catheter, incorporated herein by reference. A micro-catheter is small diameter catheter adapted for navigation is small blood vessels, which is typically introduced through a guiding catheter into the subject's body. There are competing considerations in the construction of a magnetic micro-catheter. The micro-catheter must be flexible enough for the tip to be significantly deflected in response to an applied magnetic field in order to gain access to small vessels, while also being strong enough to resist kinking that can arise when trying to navigate tight spaces and small vessels within a vasculature system.

### SUMMARY OF THE INVENTION

The present invention relates to micro-catheters, and in particular magnetically responsive micro-catheters. In one embodiment of a micro-catheter in accordance with the present invention, the flexibility of the catheter varies along its length. The micro-catheter is preferably sufficiently flexible that it can be navigated without a guidewire. The micro-catheter may be used with a guide catheter device, which serves as a conduit for delivery of the micro-catheter to the operating region. A guide catheter typically comprises a pre-shaped structures to allow easy access to specific points in the vasculature, and also provide a force support structure that allows mechanical pushing forces to insert the Guide Catheter within a subject body. The micro-catheter can be inserted into and pushed through the guide catheter into the subject's body, where it may be extended beyond the guide catheter and navigated through the vasculature system to a target destination.

Generally, various embodiments of a micro-catheter in accordance with the present invention include a proximal end and a distal end, and a lumen extending therebetween. Some embodiments of the micro-catheter have portions or regions of differing flexibility along the length of the micro-catheter, and have at least one magnetically responsive body that is responsive to an magnetic field applied to the distal end. The orientation of the distal end of the micro-catheter can be controlled with an externally applied magnetic field from one or more stationary or moving electromagnets or permanent magnets.

In one embodiment, the material and size of the distal end of the micro-catheter is sufficiently flexible and the tip is proportioned such that the tip can bend or deflect at least about 50 degrees from an initial orientation when subjected to a magnetic field of 0.08 Tesla having a reference angle 90 degrees relative to initial the orientation of the distal end of the catheter. The tip of the micro-catheter most preferably can deflect 90 degrees within about 5 mm of the distal end, which facilitates navigation in small (less than 5 mm in diameter) vessels. The distal end of the catheter is preferably capable of bending at a 4 mm radius without permanently kinking.

Some embodiments of a micro-catheter preferably have a plurality of regions of differing flexibility. Each region has successively greater flexibility from the proximal end to the distal end, as the distance between the region to distal end decreases. This varying flexibility allows a pushing force applied at the proximal end of the micro-catheter to be transmitted to the distal end without buckling the catheter, while also maintaining flexibility at the distal end to enable difficult navigation.

At least some embodiments of the micro-catheter of this invention are adapted to be introduced into the body through the micro-catheter, and can be deflected up to at least 500 in any direction upon the application of a magnetic field of less than 0.1 Tesla, and more preferably less than 0.8 Tesla. The micro-catheter stiffness is preferably sufficient to allow it to be mechanically advanced in the selected or deflected direction.

### BRIEF DESCRIPTION OF THE DRAWINGS

FIG. 1 is a side elevation view of a preferred embodiment of a micro-catheter in accordance with the principles of this invention, showing a plurality of regions of varying flexibility;

FIG. 2 is an enlarged partial longitudinal cross sectional view of the distal end portion of a micro-catheter of the preferred embodiment, showing a magnetic element disposed in the distal end portion; and

FIG. 3 is an alternate construction of the distal end of the micro-catheter in accordance with the principles of the present invention.

Corresponding reference numerals indicate corresponding parts throughout the several views of the drawings.

### DETAILED DESCRIPTION OF THE INVENTION

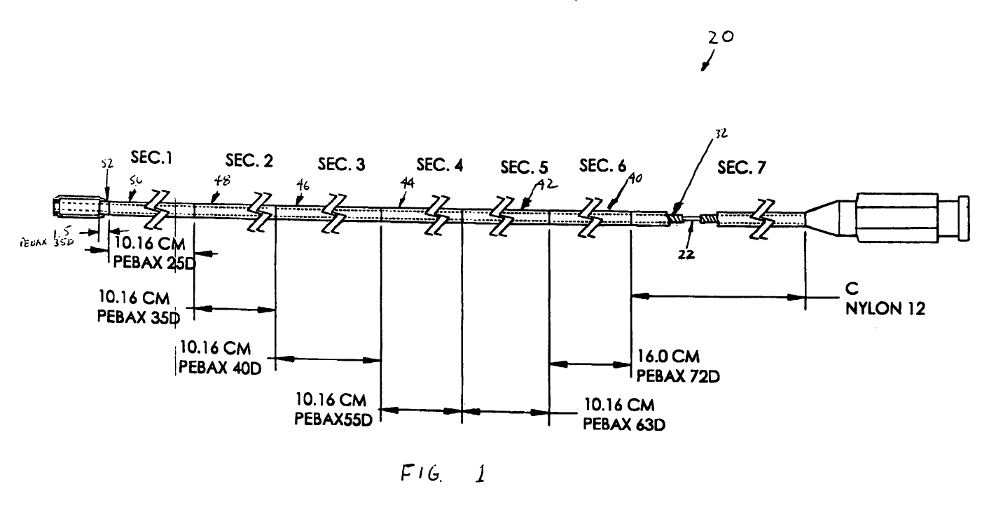

A first embodiment of a steerable, variable flexibility magnetic micro-catheter constructed according to the principles of this invention is indicated generally as 20 in FIG. 1. The micro-catheter 20 comprises a tube 22 having a proximal end 24 and a distal end 26. A lumen 28 extends substantially between the proximal end 24 and the distal end 26.

A conventional connector 30 can be mounted on the proximal end 24 of the tube 22. The tube 22 comprises an inner liner 31, which is reinforced by a support structure layer 32 surrounding the inner liner 31. In the preferred embodiment, the support structure layer 32 is comprised of an elastic wire coiled around the outer surface of the inner tube 22, but may alternatively be a sectional casing, interconnecting sleeves or other means suitable for reinforcing the inner tube 22. In this preferred embodiment, the support structure layer 32 is a 0.0007″ thick by 0.003″ wide wire having a coil pitch of about 67 wraps or turns per inch. This wire is preferable Nitinol, but could be some other suitable material.

The micro-catheter of the preferred embodiment also has regions or sections of different flexibility. In the preferred embodiment, the magnitude of the durometer of each section decreases as the position of a section approaches the distal end of the micro-catheter. The various sections comprise an individual sleeve segment having a given durometer, but may alternatively comprise a single sleeve that varies in durometer along its length. In the preferred embodiment shown in FIG. 1, there are seven sections 40, 42, 44, 46, 48 50 and 52 that each comprise a sleeve segment disposed over the coiled wire layer 32 over the inner liner 31. It should be noted that the number of sections are exemplary of the preferred embodiment, and the number, lengths, and stiffnesses of the sections may vary without departing form the principles of this invention. The sleeves have varying hardnesses ranging from 20 to 77 durometer. In the preferred embodiment, the most proximal sleeve section 40 furthest from the distal end has an approximate length in the range of 15 to 17 centimeters and a hardness in the range of 67 to 77 durometer. The sixth sleeve section 42, adjacent to sleeve 40 and distal thereto, has a length in the range of 9.66 to 10.66 centimeters and a hardness in the range of 58 to 68 durometer. The fifth sleeve section 44, adjacent to sleeve 42 and distal thereto, has a length in the range of 9.66 to 10.66 centimeters and a hardness in the range of 50 to 60 durometer. The fourth sleeve section 46, adjacent to sleeve 44 and distal thereto, has a length in the range of 9.66 to 10.66 centimeters and a hardness in the range of 35 to 45 durometer. The third sleeve section 48, adjacent to sleeve 46 and distal thereto, has a length in the range of 9.66 to 10.66 centimeters and a hardness in the range of 30 to 40 durometer. The second sleeve section 50, adjacent to sleeve 48 and distal thereto, has a length in the range of 9.66 to 10.66 centimeters and a hardness in the range of 20 to 30 durometer. The first sleeve section 52, near the distal end has a length in the range of 1 to 2 centimeters and a hardness in the range of 30 to 40 durometer. The decreasing hardness of the one or more sleeves provide the micro-catheter with an increasing degree of flexibility along its length toward the distal end of the catheter. In the preferred embodiment the sleeve sections are manufactured from Pebax or some other suitable flexible, biocompatible material or materials,

At least one of the sleeves preferably has a radiopacity effective for enabling the catheter to be viewed by fluoroscopic imaging. Preferably, the sleeve section 52 nearest the distal tip of the micro-catheter has a radiopacity suitable for viewing by a fluoroscopy or X-ray imaging device, however other or different of the sleeves can be radiopaque, as appropriate form the particular device and procedure.

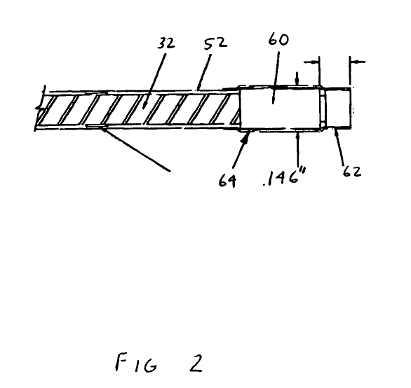

A magnetically responsive element 60 is disposed over the coiled wire layer 32 on the distal end of the micro-catheter adjacent the first sleeve section 52, as shown in FIG. 2. The magnetically responsive element or member 60 is generally tubular, and is placed over the coiled wire 32 and abuts the sleeve section 52. The magnet may be formed of a permanent magnet material such as neodymium-iron-boron (Nd—Fe—B) or other suitable magnetic material. The magnetic could also be formed of a magnetically permeable material, such as Hiperco, other suitable material. The magnetically responsive element 60 is made of such material and is of such dimensions, that under the influence of an applied magnetic field of as low as 0.1 Tesla, and more preferably as low as 0.06 Tesla, the distal end portion of the micro-catheter aligns with the local applied magnetic field direction.

In the preferred embodiment, the distal tip of the micro-catheter further comprises a sleeve tip 62 adjacent the magnetically responsive element 60. It should be noted that the sleeve tip 62 may be optionally omitted in an alternate embodiment of a micro-catheter without departing from the principles of the present invention. The sleeve tip sleeve 62 is preferably comprised of the same material as the sleeve sections, and preferably has a length in the range of 0.1 to 0.2 centimeters and a hardness in the range of 30 to 40 durometer. i.e. similar to the third section 48.

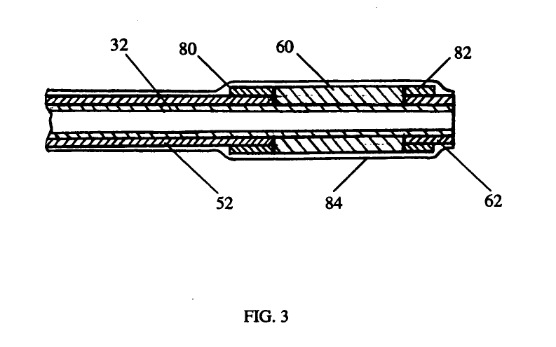

The distal end of the micro-catheter further comprises an encapsulating layer 64 surrounding the magnetically responsive element 60. The encapsulating layer 64 is preferably comprised of the same Pebax material as the sleeve sections in the form of a tube or sleeve, which is placed over the magnet sleeve 60. The hardness of the encapsulating material is preferably in the range of 58 to 68 durometer. The encapsulating layer 64 may be formed or crimped or bonded to sleeve section 52 and the sleeve tip 62 to hold the encapsulating material securely over the magnetically responsive element 60. The micro-catheter can be heated (for example by exposure to a heat stream) to cause the one or more sleeves and the encapsulating layer to refold and coalesce to form a uniform outer layer on the catheter. In an alternate embodiment, the magnet sleeve 60 may be secured in place over the coiled wire layer 32 by a pair of bracketing sleeves 80 and 82 positioned adjacent to each end of the magnetically responsive element 60, as shown in FIG. 3. In this alternate embodiment, forming or crimping of the encapsulating layer 64 may not be required.

The micro-catheter in accordance with the principles of the present invention preferably further comprises a hydrophilic layer over the outer surface of the catheter, which serves to improve the lubricity of the outer surface of the micro-catheter, as well as to seal the catheter device to reduce any toxicity from the insertion of the device into the subject's body.

In the preferred embodiment the micro-catheter has a maximum outer diameter not more than about 0.146 inches, which is sufficient for enabling use of the catheter with a typical guiding catheter.

In operation, the micro-catheter device of the present invention may be introduced through a Guiding Catheter into the subject's vasculature, and the magnetically responsive element 60 of the micro-catheter 20 is aligned by an external magnetic field to orientate the distal tip of the micro-catheter in a selected direction. The distal tip of the micro-catheter of the preferred embodiment is capable of being deflected a minimum of 50 degrees relative to the initial orientation of the distal end of the micro-catheter, when subjected to a magnetic field having a reference angle 90 degrees relative to the orientation of the distal end of the micro-catheter, wherein the magnetic field is of a magnitude of no more than about 0.1 Tesla, and more preferably no more than about 0.08, Tesla, and most preferably no more than about 0.06 Tesla. Preferably, the tip of the micro-catheter will be deflected 90 degrees when subjected to a magnetic field having a reference angle 90 degrees relative to the orientation of the distal end of the micro-catheter. The micro-catheter of the present invention is further capable of bending at a minimum radius of about 0.15 without kinking or permanently bending. Once the tip has been oriented in the selected direction, the proximal end of the micro-catheter 22 may then be pushed by hand to advance the tip though the subject body's vasculature system. The external magnetic field may be changed in orientation to realign or redirect the tip in a stepwise process to continue to steer or guide the catheter though the vasculature system until the distal end is at a selected target, such as the left coronary artery. A medical device or implant may then be inserted by the micro-catheter 20 at the distal end to the site of treatment.

The micro-catheter of the present invention will allow the surgeon to mechanically push or advance the catheter accurately and reliably in the selected direction, and enable navigation through tight small passages within the vasculature system of a patent to reach a target destination within the body. The removal of the external magnetic field will restore the flexibility to the distal end 24, which may then be realigned to another direction to further advance the catheter 20 in the body in any desired direction. Although the apparatus is described in conjunction with operations using a micro-catheter in small vessels in a subject body, it should be recognized that the inventive apparatus and techniques may be applied to other living tissues as well, or in other media, living or not, through which it may be desired to push a magnetically guided micro-catheter.

The above-described embodiments are intended to be illustrative only. For example, the mechanical pushing force applied to the proximal end of the catheter may also be provided by using a guide wire in connection with a motor that is controlled by a surgeon. There are also numerous types of magnetic surgery procedures for which the micro-catheter described and the method of controlling the micro-catheter are important. The invention can be readily adapted so that a surgeon, under guidance from an imaging system, uses the magnetic system to negotiate otherwise difficult turns and movements of the surgical device as he or she pushes a device along the interior of a small vessel. It will also be recognized that many of the inventive methods and apparatus may be used in conjunction with any coil in a non-resonant circuit that applies a magnetic force on a suspended or embedded object that is magnetically moveable. Many other modifications falling within the spirit of the invention will be apparent to those skilled in the art. Therefore, the scope of the invention should be determined by reference to the claims below and the full range of equivalents in accordance with applicable law.

What is claimed:

1. A catheter having a proximal and a distal end, the catheter comprising:
a tubular inner member;
a support structure layer that surrounds at least a portion of the tubular inner member;
a magnetically responsive member on the distal end of the inner member;
at least two sleeves axially disposed over the inner member the support structure layer, the at least two sleeves having at least two different flexibilities; and
an encapsulating layer that surrounds the magnetically responsive member on the distal end, enclosing the magnetic member on the inner member.

2. The catheter according to claim 1 wherein at least a portion of the magnet member overlies the support structure layer.

3. The catheter according to claim 1 wherein there are sleeves disposed on the proximal and distal sides of the magnetically responsive element.

4. The catheter according to claim 1 wherein there are a plurality of sleeves proximal to the magnetically responsive element, and wherein the flexibility of the sleeves increases from the proximal end of the catheter toward the magnetically responsive member

5. The catheter of claim 1, wherein the support structure comprises a reinforcing wire disposed around the tubular liner.

6. The catheter of claim 1, wherein the magnetic member comprises a sleeve of magnetic material that is axially disposed over at least a portion of the support structure layer.

7. The catheter of claim 1, wherein at least one of the at least two sleeves is comprised of a material having a radiopacity level effective for enabling the catheter to be viewed by fluoroscopic imaging.

8. The catheter of claim 1, wherein the at least two sleeves comprise a decreasing hardness as the distance to the distal end decreases.

9. The catheter of claim 8, wherein the decreasing hardness of the sleeves provides an increasing degree of flexibility toward the distal end of the catheter.

10. The catheter of claim 9, wherein the distal end of the catheter deflects at least 50 degrees relative to the longitudinal axis in response to an applied magnetic field of at least 0.1 Tesla in a direction 90 degrees from the original orientation of the distal end of the catheter.

11. The catheter of claim 1, wherein the catheter is capable of bending at a minimum radius of about 0.15 inches without kinking or permanently bending.

12. A micro-catheter having a proximal and distal end; the micro-catheter comprising:
a tubular inner member extending from the proximal end to the distal end and having a lumen therebetween;
a layer of reinforcing wire disposed around the outside of at least a portion of the inner member;
a magnetically responsive sleeve axially disposed on the inner member and disposed over at least a portion of the reinforcing wire adjacent the distal end of the micro-catheter;
a plurality of sleeves of varying lengths and stiffnesses, which are axially disposed over the inner member proximal to the magnetically responsive sleeve, the sleeves arranged in decreasing order of stiffness toward the distal end;
an encapsulating layer disposed on the distal end and surrounding at least the magnetic sleeve.

13. The micro-catheter of claim 12, wherein at least one of the plurality of sleeves are comprised of a material having a radiopacity effective for enabling the micro-catheter to be viewed by radio imaging devices.

14. The micro-catheter of claim 12, wherein the decreasing stiffness of the one or more sleeves provides an increasing degree of flexibility to the length of the micro-catheter toward the distal end of the micro-catheter.

15. The catheter of claim 13, wherein the distal end of the catheter deflects at least 50 degrees relative to the longitudinal axis in response to an applied magnetic field of at least 0.1 Tesla in a direction 90 degrees from the original orientation of the distal end of the catheter.

16. The micro-catheter of claim 15, wherein the micro-catheter is capable of bending at a minimum radius of about 0.15 inches without kinking or permanently bending.

17. The catheter of claim 12 wherein a sleeve is disposed on the proximal and distal sides of the magnetically responsive member, and wherein the encapsulation layer is bonded to the sleeves on the proximal and distal sides of the magnetically responsive member

18. The catheter of claim 16, further comprising a hydrophilic layer over at least a portion of the outer surface of the catheter.

19. The catheter device of claim 18, wherein the outside diameter of the catheter adjacent the distal end is less than about 0.146 inches, such that the catheter may be inserted within a typical guiding catheter.

20. An improved micro-catheter device having a proximal and distal end and a lumen therebetween; the improvement comprising:
a tubular inner member;
a layer of reinforcing wire disposed around the outside of the inner liner;
a magnetically responsive sleeve axially disposed over the reinforcing wire on the distal end of the inner member;
a plurality of sleeves of varying lengths and stiffnesses, which are axially disposed over the reinforcing wire layer proximal to the magnetic sleeve, wherein the plurality of sleeves are sequentially arranged in a manner such that the stiffness of the sleeves generally decreases toward the distal end; and
an encapsulating layer disposed on the distal end and surrounding the magnetic sleeve.

21. The improved micro-catheter device of claim 20, wherein the distal end of the catheter is capable of bending at a minimum radius of about 0.15 inches without kinking or permanently bending, and is capable of being deflected at least 50 degrees relative to the longitudinal axis in response to an applied magnetic field of at least 0.1 Tesla in a direction 90 degrees from the original orientation of the distal end of the catheter.

22. The improved micro-catheter device of claim 21, wherein the plurality of sleeves comprise at least 7 sleeves having a hardnesses ranging from 25 to 72 durometer.

23. The improved micro-catheter device of claim 22, wherein a first sleeve abuts the magnetic sleeve on the distal end of the micro-catheter and has a length in the range of 1 to 2 centimeters and a durometer in the range of 30 to 40 durometer.

24. The improved micro-catheter device of claim 23, wherein a second sleeve from the distal end has a length in the range of 9.66 to 10.66 centimeters and a durometer in the range of 20 to 30 durometer.

25. The improved micro-catheter device of claim 24, wherein a third sleeve from the distal end has a length in the range of 9.66 to 10.66 centimeters and a durometer in the range of 30 to 40 durometer.

26. The improved micro-catheter device of claim 25, wherein a fourth sleeve from the distal end has a length in the range of 9.66 to 10.66 centimeters and a durometer in the range of 35 to 45 durometer.

27. The improved micro-catheter device of claim 26, wherein a fifth sleeve from the distal end has a length in the range of 9.66 to 10.66 centimeters and a durometer in the range of 50 to 60 durometer.

28. The improved micro-catheter device of claim 27, wherein a sixth sleeve from the distal end has a length in the range of 9.66 to 10.66 centimeters and a durometer in the range of 58 to 68 durometer.

29. The improved micro-catheter device of claim 28, wherein a seventh sleeve from the distal end has a length in the range of 15 to 17 centimeters and a durometer in the range of 67 to 77 durometer.
